(19) United States
(12) Patent Application Publication
(10) Pub. No.: US 2026/0224393 A1
(43) Pub. Date: Aug. 6, 2026

(54) Cooling Leg Wrap Device

(71) Applicant: Shondra Thrower, North Las Vegas, NV (US)

(72) Inventor: Shondra Thrower, North Las Vegas, NV (US)

(21) Appl. No.: 19/096,789

(22) Filed: Apr. 1, 2025

Related U.S. Application Data
(60) Provisional application No. 63/753,058, filed on Feb. 3, 2025.

(51) Int. Cl. A61F 7/02; A61F 7/00

(52) U.S. Cl. CPC A61F 7/02; A61F 2007/0041; A61F 2007/0219

(57) ABSTRACT

A cooling leg wrap device is disclosed, which is a cooling device for individuals who get overheated when sleeping. The cooling leg wrap device comprises a pair of leg components, each one configured to be positioned around a user's thighs. Each leg component is an air-cushioned leg wrap made of a flexible elastic for a secure fit. Each leg component is configured with or without pockets for removable cooling gel packs. Further, a hook and loop closure secures each pocket. The leg component each comprise layers of cooling materials, comfortable cushioning, and removable ice gel packs that can provide relief for 10-12 hours before needing to be re-chilled.

### CROSS-REFERENCE TO RELATED APPLICATION

The present application claims priority to, and the benefit of, U.S. Provisional Application No. 63/753,058, which was filed on Feb. 3, 2025, and is incorporated herein by reference in its entirety.

### FIELD OF THE INVENTION

The present invention relates generally to the field of cooling leg wrap devices. More specifically, the present invention relates to a cooling device to provide long-lasting relief to the user while sleeping. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in cooling leg wrap devices. Generally, individuals often become overheated when trying to sleep, especially women experiencing menopause symptoms. Thus, at times menopausal women sometimes need to lower their body temperature to significantly reduce hot flashes.

Further, trying to sleep when overheated can be impossible and result in the individual being tired and less productive during the following day. Additionally, current products on the market, such as cooling sheets, mattresses, towels, pillows, vests, etc., do not provide long-lasting relief. Further, not having proper sleep is both frustrating and unhealthy. While the device is designed to be used by menopausal women while sleeping, it is beneficial for use by all individuals at any time.

Accordingly, there is a demand for an improved cooling leg wrap device that provides cooling for women experiencing menopausal symptoms or individuals with high fevers or who are just uncomfortable when sleeping in a hot environment. More particularly, there is a demand for a cooling leg wrap device that allows a user's body temperature to be cooled gradually and maintained for up to 12 hours before the gel packs need to be re-chilled.

Therefore, there exists a long-felt need in the art for a cooling leg wrap device that provides users with long-lasting cooling relief while sleeping. There is also a long-felt need in the art for a cooling leg wrap device that provides air-cushioned leg wraps made of a flexible elastic. Further, there is a long-felt need in the art for a cooling leg wrap device that includes pockets for removable gel packs. Moreover, there is a long-felt need in the art for a device that provides hook and loop closures to secure the device to a user's legs. Finally, there is a long-felt need in the art for a cooling leg wrap device that allows a user's body temperature to be cooled gradually and maintained for up to 12 hours before the gel packs need to be re-chilled.

The subject matter disclosed and claimed herein, in one embodiment thereof, comprises a cooling leg wrap device. The device is a cooling device for individuals who get overheated when sleeping. The cooling leg wrap device comprises a pair of leg components, each one configured to be positioned around a user's thighs. Each leg component is an air-cushioned leg wrap made of a flexible elastic for a secure fit. Each leg component is configured with or without pockets for removable cooling gel packs. Further, a hook and loop closure secures each pocket. The leg component each comprise layers of cooling materials, comfortable cushioning, and removable ice gel packs that can provide relief for 10-12 hours before needing to be re-chilled.

In this manner, the cooling leg wrap device of the present invention accomplishes all of the foregoing objectives and provides users with a device that provides cooling relief for users while sleeping. The device cools a user's body gradually and maintains cooling for up to 12 hours. The device can be worn during the day when the user is awake.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a cooling leg wrap device. The device is a cooling device for individuals who get overheated when sleeping. The cooling leg wrap device comprises a pair of leg components, each one configured to be positioned around a user's thighs. The leg components each comprise layers of cooling materials, comfortable cushioning, and removable ice gel packs that can provide cooling relief.

In one embodiment, the cooling leg wrap device is a device designed to be wrapped around a user's thighs to keep them cool at night and prevent overheating when sleeping. While the device is designed typically for women experiencing menopausal symptoms, the device is also beneficial for users with high fevers, or just for comfortable sleeping when the sleeping environment is hot. It is also contemplated that the device could be worn during the day when the user is awake. Accordingly, the cooling leg wrap device is designed to be wrapped around the user's upper thighs, delivering an initial rush of cool relief, followed by a gradual release of cooling comfort throughout the day or night.

In one embodiment, the cooling leg wrap device comprises a pair of leg components, each one configured to be positioned around a user's thighs. Typically, the leg components are cylindrical in shape with a hollow interior, allowing a user to insert their legs through the hollow interior during use. Thus, the leg components are tubed-shaped with a curved or circular sidewall defining a top perimeter and a bottom perimeter. The curved sidewall can be any suitable length as is known in the art, depending on the wants and/or needs of the user and/or length of a user's thighs, etc. Further, the top and bottom perimeter can be any suitable size as is known in the art, depending on the wants and/or needs of the user and/or size of a user's thighs, etc.

In one embodiment, each leg component is an air-cushioned leg wrap made of a flexible elastic for a secure fit. Specifically, each leg component is manufactured from an elastomeric material (i.e., elastomers), such as natural rubber, polyurethanes, lycra, polybutadiene, thermoplastic elastomers (TPE), resilin, elastin, polysulfide rubber, and elastolefin, etc., or any other suitable elastomeric materials as is known in the art. The leg components are designed to be slid over a user's legs and then stretched and pulled up over a user's thighs during use. The elastomeric material allows the leg components to be stretched wide over a user's thighs and released. Once released, the leg components then act to retain their shape, conforming to and securing the leg component in place on a user's thigh.

In one embodiment, each leg component comprises layers of cooling materials. Specifically, multiple cooling layers can be utilized, as is known in the art depending on the wants and/or needs of a user. Typically, there is an inner and outer bamboo-nylon blended fabric layers. The inner layer contacts the user's skin and thus should be soft, as well as stretchable. The outer layer should be durable and stretchable, as it can come into contact with other surfaces. Any other suitable materials can be used as is known in the art, depending on the needs and/or wants of a user for the inner and outer layers.

In one embodiment, the next inner layer (from the inner layer) is typically a thin bamboo foam padding layer to add comfort to the leg component. Any other padding layers can be utilized as is known in the art. Further, multiple padding layers can be utilized as well, depending on the needs and/or wants of a user.

In one embodiment, each leg component is configured with or without pockets. If the leg components have pockets, then the pockets are shaped and sized to retain removable cooling gel packs. Typically, a hook and loop closure secures each pocket, retaining the gel packs inside. Further, the gel packs can be frozen/chilled to provide relief for 10-12 hours before needing to be re-chilled. The gel packs allow a user's body temperature to be cooled gradually and maintained over a period of time (i.e., approximately 10-12 hours). While the device is designed to be used by menopausal women while sleeping, it is beneficial for use by all individuals at any time.

In one embodiment, an air cushion layer is included typically over the gel pack pockets. In one embodiment, the air cushion layer includes a finger air pump and release valve to allow a user to inflate or deflate the leg component, as needed. The air cushion layer is typically a bladder positioned toward a user's inner thighs, which becomes a protrusion when inflated. When the bladders are fully inflated, they typically will touch in between a user's thighs, allowing a user to comfortably sleep with the device, even on their sides. The outer bamboo-nylon blended fabric layer is then positioned over the air cushion layer to protect it and the rest of the layers of the device.

In yet another embodiment, the cooling leg wrap device comprises a plurality of indicia.

In yet another embodiment, a method of providing cooling comfort to a user while sleeping is disclosed. The method includes the steps of providing a cooling leg wrap device comprising a pair of leg components with pockets and multiple cushioning layers. The method also comprises securing the elastic leg components around a user's thighs. Further, the method comprises pumping up the air cushion layer. The method also comprises inserting frozen gel pack inserts into the leg component pockets. Finally, the method comprises providing a gradual release of cooling comfort for up to 12 hours.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the cooling leg wrap device of the present invention in accordance with the disclosed architecture;

FIG. 2 illustrates a perspective view of one embodiment of the cooling leg wrap device of the present invention showing the leg components in accordance with the disclosed architecture;

FIG. 3 illustrates a perspective view of one embodiment of the cooling leg wrap device of the present invention showing the multiple layers of cooling material in accordance with the disclosed architecture;

FIG. 4 illustrates a perspective view of one embodiment of the cooling leg wrap device of the present invention showing the removable gel cooling packs in accordance with the disclosed architecture;

FIG. 5 illustrates a perspective view of one embodiment of the cooling leg wrap device of the present invention showing the air pump and release valve in accordance with the disclosed architecture;

FIGS. 6A-C illustrate a perspective view of one embodiment of the cooling leg wrap device of the present invention showing the different components of the device in accordance with the disclosed architecture;

FIGS. 7A-B illustrate a perspective view of one embodiment of the cooling leg wrap device of the present invention showing the device in use in accordance with the disclosed architecture; and

FIG. 8 illustrates a flowchart showing the method of providing cooling comfort to a user while sleeping in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a cooling leg wrap device that provides users with long-lasting cooling relief while sleeping. There is also a long-felt need in the art for a cooling leg wrap device that provides air-cushioned leg wraps made of a flexible elastic. Further, there is a long-felt need in the art for a cooling leg wrap device that includes pockets for removable gel packs. Moreover, there is a long-felt need in the art for a device that provides hook and loop closures to secure the device to a user's legs. Finally, there is a long-felt need in the art for a cooling leg wrap device that allows a user's body temperature to be cooled gradually and maintained for up to 12 hours before the gel packs need to be re-chilled.

The present invention, in one exemplary embodiment, is a novel cooling leg wrap device. The cooling leg wrap device comprises a pair of leg components, each one configured to be positioned around a user's thighs. Each leg component is an air-cushioned leg wrap made of a flexible elastic for a secure fit. Each leg component is configured with or without pockets for removable cooling gel packs. Further, a hook and loop closure secures each pocket. The leg component each comprise layers of cooling materials, comfortable cushioning, and removable ice gel packs that can provide relief for 10-12 hours before needing to be re-chilled. The present invention also includes a novel method of providing cooling comfort to a user while sleeping. The method includes the steps of providing a cooling leg wrap device comprising a pair of leg components with pockets and multiple cushioning layers. The method also comprises securing the elastic leg components around a user's thighs. Further, the method comprises pumping up the air cushion layer. The method also comprises inserting frozen gel pack inserts into the leg component pockets. Finally, the method comprises providing a gradual release of cooling comfort for up to 12 hours. While the device is designed to be used by menopausal women while sleeping, it is beneficial for use by all individuals at any time.

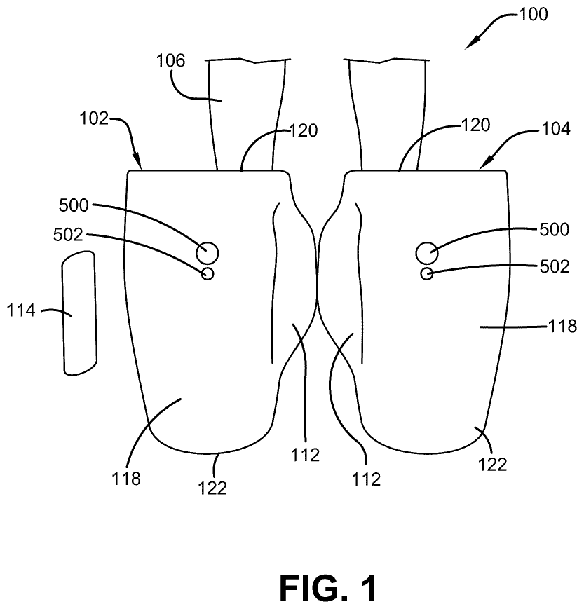

Referring initially to the drawings, FIG. 1 illustrates a perspective view of one embodiment of the cooling leg wrap device 100 of the present invention. In the present embodiment, the cooling leg wrap device 100 is an improved cooling leg wrap device 100 that provides air-cushioned leg wraps for users 106 who get overheated when sleeping or other suitable activities. Specifically, the cooling leg wrap device 100 comprises a pair of leg components 102, 104, each one configured to be positioned around a user's thighs 108. The leg components 102, 104 each comprise layers of cooling materials 110, comfortable cushioning component 112, and removable ice gel packs 114 that can provide cooling relief.

Further, the pair of leg components 102, 104 of the cooling leg wrap device 100, are cylindrical in shape with a hollow interior 116, allowing a user 106 to insert their legs through the hollow interior 116 during use. Thus, the leg components 102, 104 are tubed-shaped with a curved or circular sidewall 118 defining a top perimeter 120 and a bottom perimeter 122. The curved sidewall 118 can be any suitable length as is known in the art, depending on the wants and/or needs of the user 106 and/or length of a user's thighs 108, etc. Further, the top 120 and bottom 122 perimeter can be any suitable size as is known in the art, depending on the wants and/or needs of the user 106 and/or size of a user's thighs 108, etc.

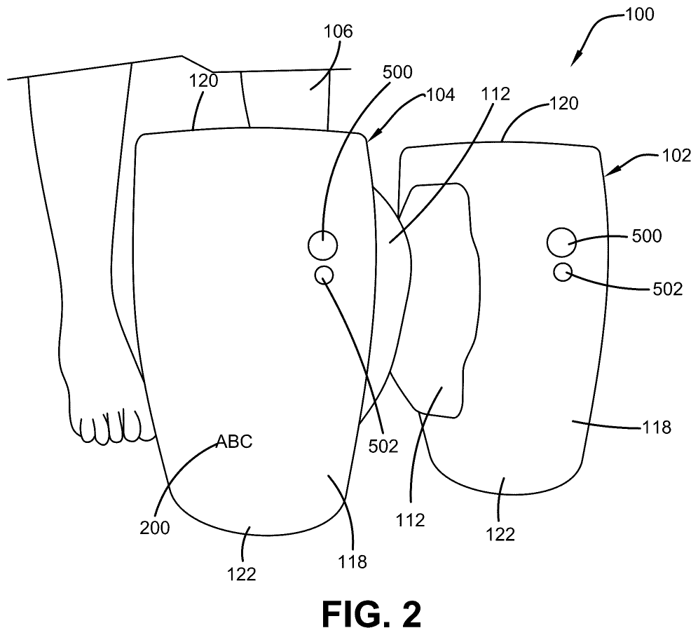

As shown in FIG. 2, each leg component 102, 104 is an air-cushioned leg wrap made of a flexible elastic for a secure fit. Specifically, each leg component 102, 104 is manufactured from an elastomeric material (i.e., elastomers), such as natural rubber, polyurethanes, lycra, polybutadiene, thermoplastic elastomers (TPE), resilin, elastin, polysulfide rubber, and elastolefin, etc., or any other suitable elastomeric materials as is known in the art. The leg components 102, 104 are designed to be slid over a user's legs and then stretched and pulled up over a user's thighs 108 during use. The elastomeric material allows the leg components 102, 104 to be stretched wide over a user's thighs 108 and released. Once released, the leg components 102, 104 then act to retain their shape, conforming to and securing the leg component 102, 104 in place on a user's thigh 108. Thus, the leg component 102, 104 provides a flexible, elastic fit.

In one embodiment, the leg component 102 or 104 is wrapped around and releasably secured around a user's thigh 108 via hook and loop fasteners, snaps, adhesives, buttons, zippers, etc., or any other suitable fastener as is known in the art.

In yet another embodiment, the cooling leg wrap device 100 comprises a plurality of indicia 200. The leg component 102, 104 of the device 100 may include advertising, a trademark, or other letters, designs, or characters, printed, painted, stamped, or integrated into the leg component 102, 104, or any other indicia 200 as is known in the art. Specifically, any suitable indicia 200 as is known in the art can be included, such as, but not limited to, patterns, logos, emblems, images, symbols, designs, letters, words, characters, animals, advertisements, brands, etc., that may or may not be cooling, air-cushioning, or brand related.

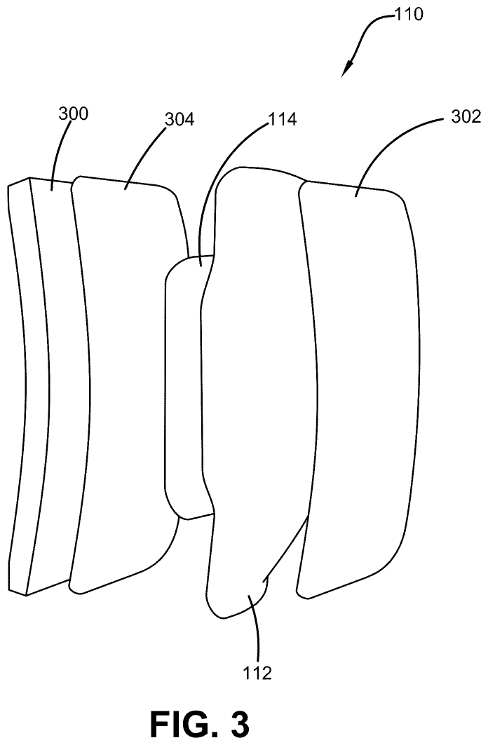

As shown in FIG. 3, each leg component 102 or 104 comprises layers of cooling materials 110. Specifically, multiple cooling layers 110 can be utilized, as is known in the art depending on the wants and/or needs of a user 106. Typically, there is an inner 300 and outer 302 bamboo-nylon blended fabric layers. The inner layer 300 contacts the user's skin and thus should be soft, as well as stretchable. The outer layer 302 should be durable and stretchable, as it can come into contact with other surfaces. Any other suitable materials can be used as is known in the art, depending on the needs and/or wants of a user 106 for the inner 300 and outer 302 layers.

In one embodiment, the next inner layer 304 (from the inner layer 300) is typically a thin bamboo foam padding layer 304 to add comfort to the leg component 102, 104. Any other padding layers 304 can be utilized as is known in the art. Further, multiple padding layers 304 can be utilized as well, depending on the needs and/or wants of a user 106.

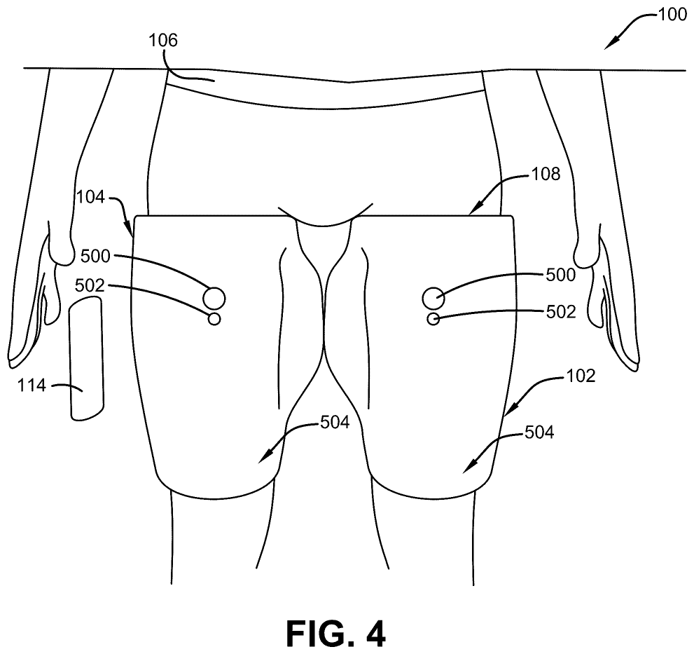

As shown in FIG. 4, each leg component 102, 104 is configured with or without pockets 400. If the leg components 102, 104 have pockets 400, then the pockets 400 are shaped and sized to retain removable cooling gel packs 114. Typically, a hook and loop closure 402 secures each pocket 400, retaining the gel packs 114 inside. Further, the gel packs 114 can be frozen/chilled to provide relief for 10-12 hours before needing to be re-chilled. The gel packs 114 allow a user's body temperature to be cooled gradually and maintained over a period of time (i.e., approximately 10-12 hours). Thus, the gel packs 114 reduce hot flashes when placed on the pulse points of the user's thighs 108.

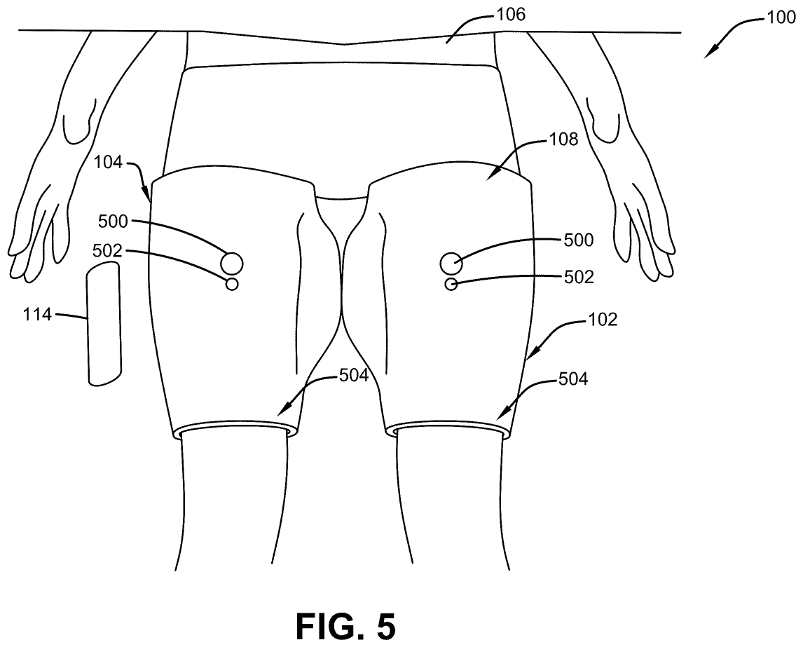

As is shown in FIG. 5, an air cushion layer (i.e., cushioning component) 112 is included typically over the gel pack pockets 400. In one embodiment, the air cushion layer 112 includes a finger air pump 500 and release valve 502 to allow a user 106 to inflate or deflate the leg component 102, 104, as needed. The air cushion layer 112 is typically a bladder positioned toward a user's inner thighs 108, which becomes a protrusion when inflated. When the bladders are fully inflated, they typically will touch in between a user's thighs 108, allowing a user 106 to comfortably sleep with the device 100, even while laying on their sides. The outer bamboo-nylon blended fabric layer 302 is then positioned over the air cushion layer 112 to protect it and the rest of the layers 300, 304 of the device 100. Further, a woven elastic tube layer 504 is provided to cover the outside of each leg component 102, 104 to provide protection and durability to the device 100.

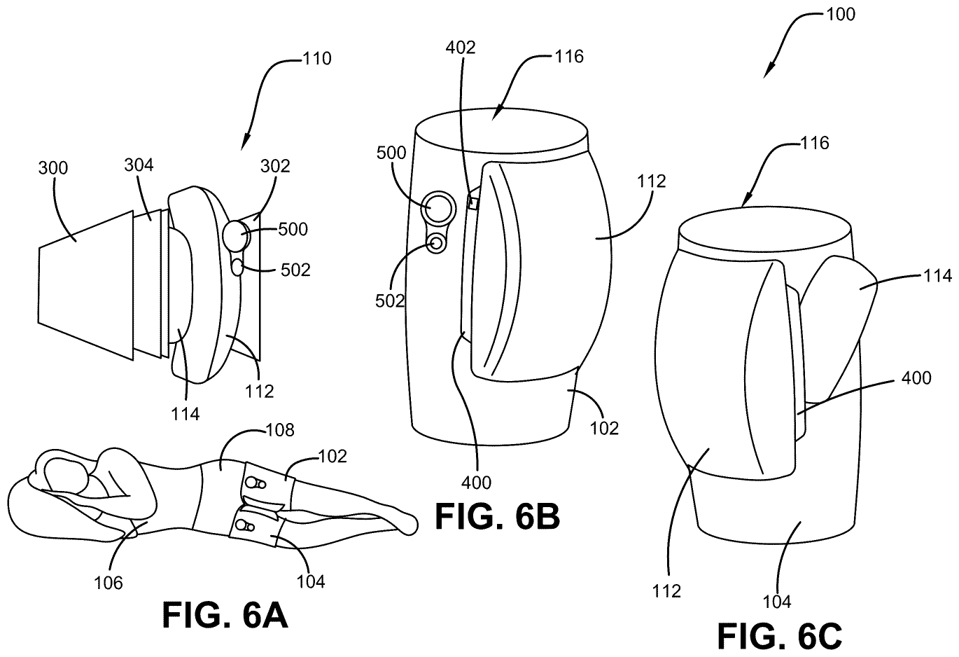

As shown in FIGS. 6A-C and 7A-B, the cooling leg wrap device 100 is a device designed to be wrapped around a user's thighs 108 to keep them cool at night and prevent overheating when sleeping. While the device 100 is designed typically for women experiencing menopausal symptoms, the device 100 is also beneficial for users 106 with high fevers, or just for comfortable sleeping when the sleeping environment is hot. It is also contemplated that the device 100 could be worn during the day when the user 106 is awake. Thus, the device 100 can be available in multiple sizes and lengths depending on the needs and/or wants of a user 106. Accordingly, the cooling leg wrap device 100 is designed to be wrapped around the user's upper thighs 108, delivering an initial rush of cool relief, followed by a gradual release of cooling comfort throughout the day or night.

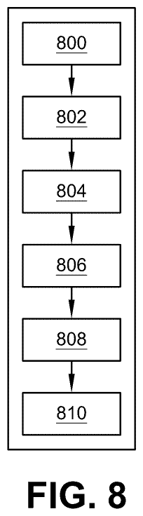

FIG. 8 illustrates a flowchart of the method of providing cooling comfort to a user while sleeping. The method includes the steps of at 800, providing a cooling leg wrap device comprising a pair of leg components with pockets and multiple cushioning layers. The method also comprises at 802, securing the elastic leg components around a user's thighs. Further, the method comprises at 804, pumping up the air cushion layer. The method also comprises at 806, inserting frozen gel pack inserts into the leg component pockets. Finally, the method comprises at 808, providing a gradual release of cooling comfort for up to 12 hours.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “cooling leg wrap device”, “cooling device”, “leg wrap device”, and “device” are interchangeable and refer to the cooling leg wrap device 100 of the present invention.

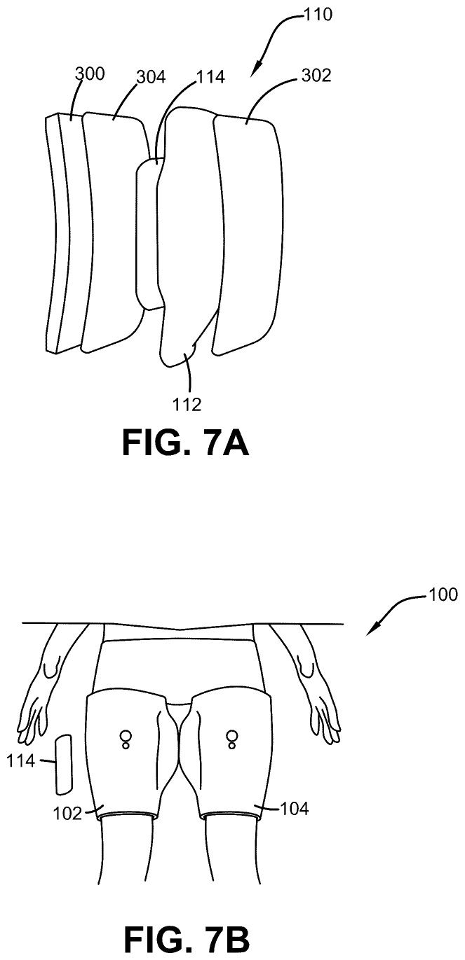

Notwithstanding the foregoing, the cooling leg wrap device 100 of the present invention can be of any suitable size and configuration as is known in the art without affecting the overall concept of the invention, provided that it accomplishes the above-stated objectives. One of ordinary skill in the art will appreciate that the cooling leg wrap device 100 as shown in FIGS. 1-8 is for illustrative purposes only, and that many other sizes and shapes of the cooling leg wrap device 100 are well within the scope of the present disclosure. Although the dimensions of the cooling leg wrap device 100 are important design parameters for user convenience, the cooling leg wrap device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cooling leg wrap device that provides air-cushioned leg wraps for users who get overheated when sleeping, the cooling leg wrap device comprising:
a pair of leg components;
wherein the pair of leg components are configured to be positioned around a user's thighs; and
further wherein the pair of leg components each comprise layers of cooling materials, an air cushion layer, and removable ice gel packs, which all provide cooling relief to a user.

2. The cooling leg wrap device of claim 1, wherein the pair of leg components are each cylindrical in shape with a hollow interior, allowing a user to insert their legs through the hollow interior during use.

3. The cooling leg wrap device of claim 2, wherein the pair of leg components are tubed-shaped with a curved sidewall defining a top perimeter and a bottom perimeter.

4. The cooling leg wrap device of claim 3, wherein each of the pair of leg components is made of a flexible elastomeric material for a secure fit, and allow the pair of leg components to be slid over a user's legs and then stretched and pulled up over a user's thighs during use.

5. The cooling leg wrap device of claim 4 further comprising a plurality of indicia.

6. The cooling leg wrap device of claim 5, wherein the layers of cooling materials comprise an inner and an outer bamboo-nylon blended fabric layer.

7. The cooling leg wrap device of claim 6, wherein the inner bamboo-nylon blended fabric layer contacts a user's skin and thus should be soft, as well as stretchable.

8. The cooling leg wrap device of claim 7, wherein the outer bamboo-nylon blended fabric layer should be durable and stretchable, as it can come into contact with other surfaces.

9. The cooling leg wrap device of claim 8, wherein the layers of cooling materials comprise a thin bamboo foam padding layer to add comfort to the pair of leg components.

10. The cooling leg wrap device of claim 9, wherein the removable ice gel packs are secured within pockets positioned on each of the pair of leg components.

11. The cooling leg wrap device of claim 10, wherein the pockets are shaped and sized to retain the removable ice gel packs and comprise a hook and loop closure to secure the pockets.

12. The cooling leg wrap device of claim 11, wherein the air cushion layer is positioned over the pockets and includes a finger air pump and release valve to allow a user to inflate or deflate the air cushion layer, as needed.

13. The cooling leg wrap device of claim 12, wherein a woven elastic tube layer is provided to cover an outside of the pair of leg components to provide protection and durability.

14. A cooling leg wrap device that provides air-cushioned leg wraps for users who get overheated when sleeping, the cooling leg wrap device comprising:
a pair of leg components which are tubed-shaped with a curved sidewall defining a top perimeter and a bottom perimeter and a hollow interior;
wherein each of the pair of leg components is made of a flexible elastomeric material for a secure fit, and allow the pair of leg components to be slid over a user's legs and then stretched and pulled up over a user's thighs during use;
wherein the pair of leg components each comprise layers of cooling materials, an air cushion layer, and removable ice gel packs, which all provide cooling relief to a user;
wherein the layers of cooling materials comprise an inner and an outer bamboo-nylon blended fabric layer;
wherein the layers of cooling materials comprise a thin bamboo foam padding layer to add comfort to the pair of leg components;
wherein the removable ice gel packs are secured within pockets positioned on each of the pair of leg components;
wherein the air cushion layer is positioned over the pockets and includes a finger air pump and release valve to allow a user to inflate or deflate the air cushion layer, as needed; and
further wherein a woven elastic tube layer is provided to cover an outside of the pair of leg components to provide protection and durability.

15. The cooling leg wrap device of claim 14, wherein the removable ice gel packs are frozen to provide relief for 10-12 hours before needing to be re-chilled, and thus allow a user's body temperature to be cooled gradually and maintained over a period of time.

16. The cooling leg wrap device of claim 14, wherein the air cushion layer is typically a bladder positioned toward a user's inner thighs, which becomes a protrusion when inflated.

17. The cooling leg wrap device of claim 14, wherein the pair of leg components are each wrapped around and releasably secured around a user's thigh via hook and loop fasteners.

18. The cooling leg wrap device of claim 14, wherein the pair of leg components can be worn during day when the user is awake.

19. The cooling leg wrap device of claim 14, wherein the cooling leg wrap device is available in multiple sizes and lengths depending on a user's needs.

20. A method of providing cooling comfort to a user while sleeping, the method comprising the following steps:
providing a cooling leg wrap device comprising a pair of leg components with pockets and multiple cushioning layers;
securing the elastic leg components around a user's thighs;
pumping up the air cushion layer;
inserting frozen gel pack inserts into the leg component pockets; and
providing a gradual release of cooling comfort for up to 12 hours.
